SEQUENCE LISTING (3 sequences)

SEQ ID NO: 1 - DNA, 20 bp, synthetic construct
  source 1..20: mol_type = other DNA
acccccatgtcagagttcct

SEQ ID NO: 2 - DNA, 19 bp, synthetic construct
  source 1..19: mol_type = other DNA
cgggcctcttcgctattac

SEQ ID NO: 3 - DNA, 20 bp, synthetic construct
  source 1..20: mol_type = other DNA
tatacaaccttgggggatgg

(19) United States
(12) Patent Application Publication
(10) Pub. No.: US 2026/0027157 A1
(43) Pub. Date: Jan. 29, 2026

(54) METHODS FOR TREATING ALZHEIMER'S DISEASE

(71) Applicant: The Regents of the University of California, Oakland, CA (US)

(72) Inventors: Stephanie Cherqui, San Diego, CA (US); Priyanka Mishra, San Diego, CA (US)

(21) Appl. No.: 18/854,500

(22) PCT Filed: Apr. 6, 2023
(86) PCT No.: PCT/US2023/017692

§ 371 Date: Oct. 4, 2024

Related U.S. Application Data
(60) Provisional application No. 63/328,574, filed on Apr. 7, 2022.

(51) Int. Cl. A61K 35/28; A61P 25/28; C07K 14/47; C07K 14/705; C12N 5/0775; C12N 9/22; C12N 15/11; C12N 15/86

(52) U.S. Cl. CPC A61K 35/28; A61P 25/28; C07K 14/4702; C07K 14/4711; C07K 14/70503; C12N 5/0662; C12N 9/226; C12N 15/111; C12N 15/86; C12N 2310/20; C12N 2510/00; C12N 2740/15043; C12N 2750/14143

(57) ABSTRACT

Methods to alleviate or treat Alzheimer's Disease or a neurological disorder or disorder, or to alleviate the symptoms of each thereof are provided, the methods comprising administering an effective amount of (HSPC) or a population of HSPCs to the subject, that are optionally gene-corrected prior to administration and that will differentiate on healthy microglia cells in the brain. The cells are capable of decreasing amyloid plaques and inflammation.

### CROSS-REFERENCE TO RELATED APPLICATION

This application claims the benefit under 35 U.S.C. § 119(e) and under the Paris Convention to U.S. Provisional Application Ser. No. 63/328,574, filed Apr. 7, 2022, the contents of which is incorporated herein by reference in its entirety.

### BACKGROUND

Alzheimer's Disease (AD) is the most prevalent cause of dementia and most common age-related neuro degenerative disorder, projected to affect over 13 million people in the APPLICANT by 2050^{1,2}. With an increasingly aging population, 3-5% of individuals 65 years or older may be suffering from AD, adding 5-7 million new cases annually³. As a result of AD, dementia develops into severe disability throughout the disease progression, resulting in death generally within 5-12 years after onset⁴. Significant neuronal degradation, inflammation and progressive memory and behavioral decline results from an accumulation of extracellular β-amyloid (Aβ) plaque and hyperphosphorylated tau in neurofibrillary tangles (NFT) throughout the brain, predominantly in hippocampus and cortex. These major hallmarks of AD lead to increased oxidative stress, inflammation, synapse loss, and cholinergic dysfunction, and elicit neuronal dystrophy⁵. Current treatments approved by FDA assist in management of cognitive impairment, yet there are no effective disease-modifying treatments available^{5,6}. Given the complex pathobiology of AD, any potential treatment must target multiple pathological pathways to improve the disease state.

### SUMMARY OF THE DISCLOSURE

For a long time, reactive microglia have been considered a consequence of AD pathology, however they are now regarded as potentially playing a role in disease progression and may be initiation^{25,27,29,34}. However, the roles of microglia in AD are still a matter of intense debate (McGeer et al., Neurology 42 (1992)).

Applicant has shown herein direct evidence that microglia play a key role in Alzheimer's disease progression and that replacing diseased App/Psen1 microglia with healthy ones via single WT HPSC transplantation in 5xFAD mice led to complete rescue of the neurocognitive impairment in the mice.

Thus, provided herein is a method to alleviate or treat Alzheimer's Disease. The method comprises, or consisting essentially or, or consisting of, administering to a subject in need thereof either systemic or in the hippocampus of the subject a hematopoietic stem or progenitor cell (HSPC) or a population of HSPCs to the subject, that are optionally gene-corrected prior to administration and that will differentiate on healthy microglia cells in the brain. The cells are capable on decreasing amyloid plaques and inflammation, thereby alleviating or treating AD.

Also provided are methods and compositions for using CD34⁺ HSPC gene therapy to correct known familial mutations in AD.

The methods and compositions are shown using the 5xFAD double transgenic mouse model, which expresses mutant human amyloid beta (A4) precursor protein 695 (APP) with the Swedish (K670N, M671L), Florida (I716V), and London (V7171) Familial Alzheimer's Disease (FAD) mutations and human presenilin 1 (PS1) harboring two FAD mutations (M146L and L286V) results in robustly increased Aβ plaque accumulation in the hippocampus and cortex by 2 to 4 months of age^{18,19} was used. These mice exhibit significant neurocognitive impairment and reduced anxiety behavior between 3 to 6 months of age²⁰. Moreover, 5xFAD mice demonstrate major features of amyloid plaque pathology of AD, including dysfunctional microglia^{18,21}.

Applicant provides herein that single systemic wild-type HSPC transplantation into adult 5xFAD mice led to the preservation of memory and neurocognitive performance, and to the reduction of the Aβ plaque burden in hippocampus and cortex. Donor or donor-derived HSPCs differentiated into microglia with active amyloid plaque clearance potential while also leading to the reduction of neuroinflammation. This work opens new therapeutic avenues using HSPC gene therapy for the treatment of AD.

Further provided herein is a method that utilizes CD34⁺ HSPC transplantation to microglia in the brain and spinal cord, and into macrophages in the DRGs, to preserve of neurons and locomotor function¹³. WT HSPC transplantation can result in the generation of healthy microglia that could lead to the reduction of neuroinflammation and Aβ plaque build-ups in the most affected areas of the brain in AD. Alternatively, the use of the patient's own hematopoietic stem and progenitor cells (HSPCs) are provided for use in a method comprising, or consisting essentially of, or consisting of an introduced or corrected gene involved in AD pathogenesis is a treatment for Alzheimer's Disease in a patient by transplanting the gene-modified HSPCs either systemically or locally within the hippocampus in the patient. The genes to be corrected are PP, MAPT, PSEN1, PSEN2 and Trem2.

### BRIEF DESCRIPTION OF THE FIGURES

FIGS. 1A-1L: Transplantation of WT HSPCs prevents neurocognition impairments in 5xFAD mice. (FIG. 1A) Schematic representation of the experimental design and timeline. (FIGS. 1B-1D) Memory recognition test evaluated by discrimination index (FIG. 1F) and preference index (FIG. 1C) in mice at 6 months of age. A representative tracking plot of the 2-day test are shown for 5xFAD/5xFAD HSPC and 5xFAD/WT HSPC mice (FIG. 1D). (FIGS. 1E-1H) Elevated plus maze test in the different mouse groups with time expressed in seconds(s) spent in the closed arms (FIG. 1E), and open arms (FIG. 1F), and the ratio of time spent in the open arms versus total time of the test (FIG. 1G). A representative tracking plot of the test is shown for 5xFAD/5xFAD HSPC and 5xFAD/WT HSPC mice (FIG. 1H). (FIGS. 1I-1L) Open field test with distance expressed in meters (m) covered by the different mouse groups in the periphery (FIG. 1I), corners (FIG. 1J), and total distance traveled (FIG. 1K). A representative tracking plot of the test is shown for 5xFAD/5xFAD HSPC and 5xFAD/WT HSPC mice (FIG. 1L). All data are indicated as mean±s.e.m. **P<0.005 and ***P<0.0005 determined as one-way ANOVA followed by Tukey's multiple comparisons.

FIGS. 2A-2K: Transplantation of WT HSPCs led to the decrease of Aβ plaque deposition in the cortex and hippocampus in 5xFAD mice. (FIGS. 2A-2D) Representative sagittal sections of the cortex (FIG. 2A, FIG. 2C) and hippocampus (FIG. 2B, FIG. 2D) stained against the Aβ plaque marker 6E10 of 5xFAD/5xFAD HSPC (FIG. 2A, FIG. 2B) and 5xFAD/WT HSPC (FIG. 2C, FIG. 2D) mice. Scale bars, 500 μm. Quantification of the plaque number (FIG. 2E, FIG. 2H), area occupied by the plaques (FIG. 2F, FIG. 2I), and percentage of total area (FIG. 2G, FIG. 2J) occupied in the cortex (FIGS. 2E-2G) and hippocampus (FIGS. 2H-2J). (FIG. 2K) Triton-X-soluble and guanidine insoluble of Aβ₁₋₄₂ (ELISA) in the brain of WT (n=3), 5xFAD (n=3), 5xFAD/5xFAD HSPC (n=3) and 5xFAD/WT HSPC (n=3) mice. All data are indicated as mean±s.e.m. **P<0.005, ***P<0.0005 and ****P<0.0001 determined as one-way ANOVA, followed by Tukey's multiple comparisons.

FIGS. 3A-3H: Infiltration of transplanted HSPCs into the brain of 5xFAD mice and differentiation into microglia leads to microglia activation reduction and Aβ plaque engulfment. (FIG. 3A) Representative immunohistochemistry image of a sagittal section of the brain from 5xFAD/WT HSPC mice at 4-month post-transplantation showing GFP⁺ cells, insets showing GFP⁺ cells in hippocampus and cortex. Scale bars, 100 μm (a, insets) (FIG. 3B) Representative images of hippocampus sections immunostained for the microglial marker Iba1, inset showing dentate gyrus region of the hippocampus. Scale bars, 100 μm. (FIG. 3C, FIG. 3D) Quantification of the area occupied by of Iba1 cells in hippocampus and cortex. Data are means±s.e.m. **P<0.005, ***P<0.0005, and ****P<0.0001 determined as one-way ANOVA, followed by followed by Tukey's multiple comparisons. (FIG. 3E) Representative chromogenic image showing Iba1⁺ microglia (green) and 6E10⁺ plaques (purple). Scale bars, 50 μm. (FIG. 3F) Representative 3D reconstitution of immunofluorescence image of brain sections from 5xFAD/5xFAD HSPC (upper panel) and 5xFAD/WT HSPC mice (lower panel), from left to right, stained with anti-GFP, anti-Iba1 and anti-6E10⁺ antibodies. Merged image on the extreme right. Scale bars, 10 μm or 2 μm (insets). (FIG. 3G) inset showing inflamed active Iba1⁺ in close proximity to 6E10⁺ plaques a 5xFAD/5xFAD HSPC mouse. (FIG. 3H) Inset showing GFP″ Iba1⁺ microglia engulfing 6E10⁺ plaques engulfed within GFP+.

FIGS. 4A-4B: RNA-seq revealed a shift in microglia and endothelial transcriptome profile in cortex and hippocampus, respectively, after WT HSPC transplantation in 5xFAD. (FIG. 4A) Barplots and violin plots for composite gene score analysis of disease-associated microglia (DAM) stage 2 and neurodegeneration associated endothelial cells gene set in cortex and hippocampus of WT (C), 5xFAD/WT HSPC (T), 5xFAD (D), 5xFAD/5xFAD HSPC (M). Significant P values (P<0.05) between T versus D are indicated as determined by Welchs' Two Sample t-test with Benjamini-Hochberg correction. (FIG. 4B) Heat map showing the top 60 differentially expressed genes in cortex and hippocampus. Barplots and violin plots showing 24 microglia-related in cortex and 7 endothelial-related significantly differentially expressed genes in the hippocampus, respectively, between 5xFAD/WT HSPC and 5xFAD brains. Significant P values (P<0.05) between T versus D are indicated as determined by Welchs' Two Sample t-test with Benjamini-Hochberg correction. The numbers on the top-right corner specify ROC-AUC values of T, D, and M compared to C respectively.

FIGS. 5A-5B. Infiltration of transplanted HSPCs into the brain of 5xFAD mice and differentiation into microglia leads to microglia activation reduction and Aβ plaque engulfment. (FIG. 5A) Representative images of cortex sections immunostained for the microglial marker Iba1. Scale bars, 100 μm. (FIG. 5B) Image showing GFP⁺ Iba1⁺ microglia engulfing 6E10⁺ plaques and their orthogonal view of brain section from a 5xFAD mouse transplanted with WT HSPCs, stained with anti-GFP, anti-Iba1 and anti-6E10 antibodies (grey scaled, see FIG. 3F for grey scale comparison of antibody staining). Scale bars, 10 μm. Inset shows colocalization of GFP⁺ Iba1⁺ with 6E10⁺ plaque. Scale bars, 2 μm.

FIGS. 6A-6B: Violin plots for composite gene score analysis of disease-associated microglia (DAM) stage 1 and universal macrophage marker gene set in cortex and hippocampus of WT (C), 5xFAD/WT HSPC (T), 5xFAD (D), 5xFAD/5xFAD HSPC (M). Significant P values T versus D are indicated. (FIG. 6A) shows disease associated microglia (stage 1). (FIG. 6B) shows universal macrophage marker. The numbers on the top right corner specify ROC AUC values of T, D, and M compared to C, respectively.

### DETAILED DESCRIPTION

Throughout this disclosure, various publications, patents and published patent specifications are referenced by an identifying citation. The disclosures of these publications, patents and published patent specifications are hereby incorporated by reference in their entirety into the present disclosure.

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

### Definitions

As used in the specification and claims, the singular form “a”, “an” and “the” include plural references unless the context clearly dictates otherwise. For example, the term “a cell” includes a plurality of cells, including mixtures thereof.

| As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

The term “isolated” as used with respect to cells, in particular stem cells, such as mesenchymal stem cells, refers to cells separated from other cells or tissue that are present in the natural tissue in the body.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Pat. Nos. 4,683,195 and 4,683,202 and Innis et al. (1990) Mol. Cell Biol. 10(11):5977-5982 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype” refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address:

http://www.ncbi.nlm.nih.gov/blast/Blast.cgi, last accessed on May 21, 2008. Biologically equivalent polynucleotides are those having the specified percent homology and encoding a polypeptide having the same or similar biological activity.

The term “an equivalent nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, or nucleic acid-nucleic acid in nature.

“Hybridization” refers to a reaction in which one or more polynucleotides react to form a hybridization complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in about 10 x SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “mismatches” refers to hybridized nucleic acid duplexes which are not 100% homologous. The lack of total homology may be due to deletions, insertions, inversions, substitutions or frameshift mutations.

As used herein, the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double-and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

A “composition” typically intends a combination of the active agent, e.g., the stem cell or population thereof, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include into the hippocampus, intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

An agent of the present disclosure can be administered for therapy by any suitable route of administration. It will also be appreciated that the optimal route will vary with the condition and age of the recipient, and the disease being treated.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

The term “express” refers to the production of a gene product. In some embodiments, the gene product is a polypeptide or protein. In some embodiments, the gene product is an mRNA, a tRNA, an rRNA, a miRNA, a dsRNA, or a siRNA.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski, et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, CA) and Promega Biotech (Madison, WI). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in M. MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optionally contain detectable labels and are exemplified and described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue.™., and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are known in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarry system that can dispense from 12 to 64 spots onto multiple glass slides (Intelligent Bio-Instruments); Molecular Biology Workstation and NanoChip (Nanogen, Inc.); a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarrays” are also described in U.S. Patent Publication Nos.: 2007/0111322; 2007/0099198; 2007/0084997; 2007/0059769 and 2007/0059765 and U.S. Pat. Nos. 7,138,506; 7,070,740 and 6,989,267.

In one aspect, “gene chips” or “microarrays” containing probes or primers homologous to a polynucleotide described herein are prepared. A suitable sample is obtained from the patient, extraction of genomic DNA, RNA, protein or any combination thereof is conducted and amplified if necessary. The sample is contacted to the gene chip or microarray panel under conditions suitable for hybridization of the gene(s) or gene product(s) of interest to the probe(s) or primer(s) contained on the gene chip or microarray. The probes or primers may be detectably labeled thereby identifying the sequence(s) of interest. Alternatively, a chemical or biological reaction may be used to identify the probes or primers which hybridized with the DNA or RNA of the gene(s) of interest. The genotypes or phenotype of the patient is then determined with the aid of the aforementioned apparatus and methods.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein, e.g. a calcium handling protein, a t-tubule protein or alternatively, a calcium pump protein. In another aspects, the substantially homogenous population have a decreased (e.g., less than about 95%, or alternatively less than about 90%, or alternatively less than about 80%, or alternatively less than about 75%, or alternatively less than about 70%, or alternatively less than about 65%, or alternatively less than about 60%, or alternatively less than about 55%, or alternatively less than about 50%) of the normal level of expression than the wild-type counterpart cell or tissue.

A “neurodegenerative disease” is a condition in which cells of the brain and spinal cord are lost. Examples of neurodegenerative diseases include, but are not limited to Alzheimer's Disease.

An “effective amount” is an amount sufficient to effect beneficial or desired results. An effective amount can be administered in one or more administrations, applications or dosages and can be empirically determined by those of skill in the art.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

A “control” is an alternative subject or sample used in an experiment for comparison purpose. A control can be “positive” or “negative”. For example, where the purpose of the experiment is to determine a correlation of a mutated allele with a particular phenotype, it is generally preferable to use a positive control (a sample from a subject, carrying such mutation and exhibiting the desired phenotype), and a negative control (a subject or a sample from a subject lacking the mutated allele and lacking the phenotype).

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, WI. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4.

A “mesenchymal stem cell” or MSC, is a multipotent stem cell that can differentiate into a variety of cell types. The designation MSC also refers to the term “marrow stromal cell”. Cell types that MSCs have been shown to differentiate into in vitro or in vivo include osteoblasts, chondrocytes, myocytes, and adipocytes. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. While this is an accurate description for one function of MSCs, the term fails to convey the relatively recently-discovered roles of MSCs in repair of tissue.

A hematopoietic stem progenitor stem and progenitor cell (HSPC) intends a precursor cell that possesses the capacity for self-renewal and multilineage differentiation. In the bone marrow (BM), HSPCs warrant blood cell homeostasis. In addition, they may also replenish tissue-resident myeloid cells and directly participate in innate immune responses once they home to peripheral tissues. The cells can be isolated from mobilized peripheral blood or umbilical cord blood. See, e.g., Bujko et al. (2019) “Hematopoietic Stem and Progenitor Cells (HSPCs)” Adv. Exp. Med. Biol. Adv Exp Med Biol. 2019; 1201:49-77. doi: 10.1007/978-3-030-31206-0_3. PMID: 31898781. The cells are characterized by the marker profile CD34⁺. Methods to expand and culture such cells are known in the art See, e.g., Yadav et al. (2020), Int. J. Stem Cells, Vol. 13(3):326-334, available at

https://www.nebi.nlm.nih.gov/pmc/articles/PMC7691860/.

A “neural or neuronal stem cell” as used herein refers to a cell that has the ability to self-replicate and give rise to multiple specialized cell types of the nervous system. In some aspect, a neural stem cell is a multipotential neural stem Ycell in the subventricular zone (SVZ) of the forebrain lateral ventricle (LV).

A clone or “clonal population” is a line of cells that is genetically identical to the originating cell; in this case, a stem cell. A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a farther stage of cell differentiation. Progenitor cells are often found in adult organisms, they act as a repair system for the body. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, an ependymal cell and a neural stem cell from the forebrain lateral ventricle (LV).

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

As used herein, the “lineage” of a cell defines the heredity of the cell, i.e. its predecessors and progeny. The lineage of a cell places the cell within a hereditary scheme of development and differentiation.

A derivative of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells. In one aspect, this cell or population is described as “donor-derived.”

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi K. et al. (2007) Cell advance online publication 20 Nov. 2007; Takahashi K. & Yamanaka S. (2006) Cell 126:663-76; Okita K. et al. (2007) Nature 448:260-262; Yu, J. et al. (2007) Science advance online publication 20 Nov. 2007; and Nakagawa, M. et al. (2007) Nat. Biotechnol. Advance online publication 30 Nov. 2007.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A population of cells intends a collection of more than one cell that is identical (clonal) or non-identical in phenotype and/or genotype.

Amyloid beta precursor protein (APP), is an integral membrane protein expressed in many tissues and concentrated in the synapses of neurons. It functions as a cell surface receptor and has been implicated as a regulator of synapse formation, neural plasticity, antimicrobial activity, and iron export. APP is the precursor molecule whose proteolysis generates amyloid beta (Aβ), a polypeptide containing 37 to 49 amino acids residues, whose amyloid fibrillar form is the primary component of amyloid plaques found in the brains of Alzheimer's disease patients. The protein sequence can be found at UniProt P05067 (human) and P12023 (mouse), or NCBI Ref. NP000745 (human), NP001129488 (human) or NP001185752 (murine) or NP001185753 (murine). mRNA encoding the protein can be found at NCBI Ref. NM201414 or NM000484 (human) or NM001198823 or NM001198824 (murine), all last accessed on Apr. 1, 2023).

Microtubule associated protein tau (MAPT) gene provides instructions for making a protein called tau that is found throughout the nervous system, including in nerve cells (neurons) in the brain. It is involved in assembling and stabilizing microtubules, which are rigid, hollow fibers that make up the cell's structural framework (the cytoskeleton). Microtubules help cells maintain their shape, assist in the process of cell division, and are essential for the transport of materials within cells Six different versions (isoforms) of the tau protein are produced in the adult brain The isoforms vary in length from 352 to 441 protein building blocks (amino acids). A region of the protein called the microtubule-binding domain, which is the part of the protein that attaches (binds) to microtubules, also varies among the isoforms In three of the isoforms, the microtubule-binding domain contains three repeated segments. In the other three isoforms, this domain contains four repeated segments. Typically, the brain has approximately the same amount of three-repeat isoforms and four-repeat isoforms. This balance appears to be essential for the normal function of neurons. See https://medlineplus.gov/genetics/gene/mapt/, last accessed on Apr. 1, 2023.

The gene that encodes MAPT transcript undergoes complex, regulated alternative splicing, giving rise to several mRNA species. MAPT transcripts are differentially expressed in the nervous system, depending on stage of neuronal maturation and neuron type. MAPT gene mutations have been associated with several neurodegenerative disorders such as Alzheimer's disease, Pick's disease, frontotemporal dementia, cortico-basal degeneration and progressive supranuclear palsy. Amino acid and polynucleotide sequence information can be found at https://www.ncbi.nlm.nih.gov/gene/4137, last accessed on Apr. 1, 2023.

Presenilin 1 (PSEN1) protein is one part (subunit) of a complex called gamma-(γ-) secretase. Presenilin 1 carries out the major function of the complex, which is to cut apart (cleave) other proteins into smaller pieces called peptides. This process is called proteolysis, and presenilin 1 is described as the proteolytic subunit of γ-secretase. The γ-secretase complex plays a role in processing amyloid precursor protein (APP), which is made in the brain and other tissues. γ-secretase cuts APP into smaller peptides, including soluble amyloid precursor protein (sAPP) and several versions of amyloid-beta (B) peptide. Evidence suggests that sAPP has growth-promoting properties and may play a role in the formation of nerve cells (neurons) in the brain both before and after birth. Other functions of sAPP and amyloid-β peptide are under investigation. See https://medlineplus.gov/genetics/gene/psen1/. The amino acid and polynucleotide sequence for the protein and gene can be found at https://www.ncbi.nlm.nih.gov/nuccore/?term=Presenilin+1+(PSEN1), last accessed at Apr. 1, 2023.

Triggering receptor expressed on myeloid cells 2 (Trem2) is a protein that that in humans is encoded by the TREM2 gene. It is expressed on macrophages, immature monocyte-derived dendritic cells, osteoclasts, and microglia, which are immune cells in the central nervous system. In the liver, TREM2 is expressed by several cell types, including macrophages, that respond to injury. In the intestine, TREM2 is expressed by myeloid-derived dendritic cells and macrophage. TREM2 is overexpressed in many tumor types and has anti-inflammatory activities. The amino acid and polynucleotide sequence for the protein and gene can be found at

https://www.ncbi.nlm.nih.gov/nuccore/?term=Triggering+receptor+expressed+on+myeloid+cells+2+(Trem2), last accessed at Apr. 1, 2023.

### MODES FOR CARRYING OUT DISCLOSURE

Applicant provides methods for treating a neurodegenerative disease, disorder or a symptom thereof in a subject in need thereof, wherein the neurodegenerative disease, disorder or symptom is selected from the group of: alleviating or treating Alzheimer's Disease (AD) related to microglia inflammation; reducing Aβ plaque burden in the hippocampus and cortex; promoting or differentiating microganglia; promoting the differentiation of a donor hematopoietic stem or progenitor cell or a donor cell population comprising the hematopoietic stem or progenitor cell into microglia; or reducing neuroinflammation; by administering to the subject an effective amount of a hematopoietic stem or progenitor cell (HSPC) or a population of HSPCs to the subject, thereby treating the neurodegenerative disease, disorder or symptom in the subject. The HSPC or population thereof are in one aspect “donor” or donor-derived HSPC or a population thereof in that they are delivered or donated to the subject.

As used herein, the term “neurodegenerative disease or disorder” intends a condition in which cells of the brain and spinal cord are lost. Examples of neurodegenerative diseases include Alzheimer's Disease, Parkinson's disease, prion disease, amyotrophic lateral sclerosis, motor neuron disease, Huntington's disease, spinocerebellar ataxia and dementia. To alleviate or treat the disease intends a reduction or in some aspects, an eradication of the clinical markers or hallmarks of the disease. To treat the neurodegenerative symptoms intends a physical or mental problem that a subject experiences which may indicate that the subject has or is suffering from the disease or condition. Symptoms cannot be seen and do not show up on medical tests. Non-limiting examples of symptoms of a neurological disease include dementia, memory loss, balance, numbness, or other cognitive decline.

Provided herein is a method to alleviate or treat Alzheimer's Disease (AD) related to microglia inflammation in a subject in need thereof, comprising, or consisting essentially of, or consisting of, administering a hematopoietic stem or progenitor cell (HSPC) or a population of HSPCs to the subject, thereby alleviating or treating the AD related to microglia inflammation. In one aspect, an effective amount of the HSPC or population thereof is administered.

Alzheimer's Disease (AD) is the most prevalent cause of dementia and most common age-related neurodegenerative disorder. As a result of AD, dementia develops into severe disability throughout the disease progression, resulting in death generally within 5-12 years after onset. Symptoms of AD can include neuronal degradation, inflammation and progressive memory and behavioral decline that result from an accumulation of extracellular β-amyloid (Aβ) plaque and hyperphosphorylated tau in neurofibrillary tangles (NFT) throughout the brain, predominantly in hippocampus and cortex. These major hallmarks of AD lead to increased oxidative stress, inflammation, synapse loss, and cholinergic dysfunction, and elicit neuronal dystrophy.

As is apparent to the skilled artisan, the therapeutic method of this disclosure can be combined with diagnostic methods for an initial assessment of the subject to be treated and thereafter, to determine if the therapy was successful. Symptoms that can be used for initial assessment and to monitor therapy include changes in personality or behaviors, memory impairment; cognitive decline, mental status steps, neuropsychological tests, and laboratory tests, such as examination of Amyloid and tau proteins can be measured in the cerebrospinal fluid. The ratio of these proteins can help determine whether Alzheimer's is present. Alzheimer's disease can also lead to the progressive loss of brain cells, which can be evaluated with a brain scan such as magnetic resonance imaging (MRI), computerized tomography (CT), and positron emission tomography (PET). A PET scan uses a radioactive substance known as a tracer to detect substances in the body. There are different types of PET scans. The most commonly used PET scan is a fluorodeoxyglucose (FDG) PET scan. This scan can identify brain regions with decreased glucose metabolism. The pattern of metabolism change can distinguish between different types of degenerative brain disease. PET scans have recently been developed that detect clusters of amyloid proteins (plaques) or tau (neurofibrillary tangles), which are associated with Alzheimer's dementia. These types of PET scans are typically used in the research setting.

In another aspect the HSPC or the population of HSPCs is wild-type. In another aspect the cells are CD34⁺ HSPCs. In other aspect, the HSPC or a population of HSPCs is genetically modified to correct genes tied to the neurodegenerative disorder. The cells in the population can be substantially homogenous HSPC or a clonal population, e.g., CD34⁺ HSPCs. They can be autologous or allogenic to the subject being treated. Methods to prepare substantially homogenous or clonal populations are known in the art.

In another aspect, the population of the HSPC cells comprises at least 80%, or at least 90%, or at least 95%, or at least 97%, of the cells of the population are CD34+ HSPCs.

The HSPC or population thereof can be administered as a composition, with one or more pharmaceutically acceptable carriers.

In a further aspect, the HSPC or a population of HSPCs is autologous or allogeneic to the subject.

In one aspect, the Alzheimer's Disease is due to mutations in known causative genes, optionally amyloid beta precursor protein (APP), microtubule associated protein tau (MAPT), the gene encoding presenilin 1 (PSEN1) or triggering receptor expressed on myeloid cells 2 (Trem2). In another aspect, the HSPC or population of HSPCs are modified to correct for known familial mutations such as in the APP, MAPT, PSEN1 and TREM2. The gene correction can occur by a method selected from gene addition, optionally by use of a vector, such as an adeno-associated viral vector or a lentiviral vector, CRISPR/Cas9 technology, or by prime editing—correction of single base pair in a the defective gene.

In the embodiment, the HSPC comprises one or more of an exogenous or wild-type gene selected from amyloid beta precursor protein (APP), microtubule associated protein tau (MAPT), the gene encoding presenilin 1 (PSEN1) or triggering receptor expressed on myeloid cells 2 (Trem2). The cells can be modified any method known in the art, such as those described herein.

In one aspect of the methods, the method alleviates disorientation, loss of bodily function, neuroinflammation or cognitive impairment.

In some aspect, the cells or the genes modified in the cells, e.g., amyloid beta precursor protein (APP), microtubule associated protein tau (MAPT), the gene encoding presenilin 1 (PSEN1) or triggering receptor expressed on myeloid cells 2 (Trem2) are detectably labeled.

The cells or population of cells can be administered systemically or locally. In one aspect, the HSPC or population of HSPC are administered through the hippocampus. In another aspect they are administered systemically. The cell or population of cells can be combined with a carrier such as a pharmaceutically acceptable carrier for the purpose and route of administration. The dose, dosing schedule and route of administration will be determined by the treating physical or veterinarian and can be determined taking into account the health, age and gender of the subject.

The subjects to be treated can be mammals, e.g., a murine, canine, feline, bovine, equine or a human patient. When the subject is a non-human mammal, the method is useful for the treatment of the non-human mammal or for use as an animal model to test new combination therapies. As is apparent to the skilled artisan, a control animal not receiving the treatment can be used as a point of reference. The methods can be combined with other treatments for neurological disorders like Alzheimer's Disease.

Applicant provides a methods for treating or alleviating the symptoms of AD in a subject in need thereof, by administering to the subject an effective amount of a hematopoietic stem or progenitor cell (HSPC) or a population of HSPCs to the subject. The HSPC or population thereof are in one aspect “donor” or donor-derived HSPC or a population thereof in that they are delivered or donated to the subject. Administration can be local or systemic. The cells can be autologous or allogeneic to the subject being treated and can be wild-type HSPC. The HSPC or population are, in one aspect, CD34+ and can optionally be delivered in a pharmaceutically acceptable carrier. When a population is administered, it can be substantially homogenous or clonal for the HSPC or CD34+ HSPC. The method can be combined with other therapeutic or diagnostic methods known in the art or as described herein. One of skill in the art can monitor the therapy and based on clinical or observational criteria, evaluate when the therapy has been effective. Effective therapy will differ with the subject being treated and the purpose of the therapy.

Applicant provides a methods for treating or alleviating the symptoms of AD in a human or mammal in need thereof, by administering to the subject an effective amount of a hematopoietic stem or progenitor cell (HSPC) or a population of HSPCs to the human or mammal, respectively. The HSPC or population thereof are in one aspect “donor” or donor-derived HSPC or a population thereof in that they are delivered or donated to the human or mammal. Administration is systemic and the cells are autologous to the human or mammal being treated. The HSPC or population are CD34+ and can optionally be delivered in a pharmaceutically acceptable carrier. When a population is administered, it can be substantially homogenous or clonal for the HSPC or CD34+ HSPC. The method can be combined with other therapeutic or diagnostic methods known in the art or as described herein. One of skill in the art can monitor the therapy and based on clinical or observational criteria, evaluate when the therapy has been effective. Effective therapy will differ with the human or mammal t being treated and the purpose of the therapy.

Applicant provides methods for reducing Aβ plaque burden in the hippocampus and cortex by administering to the subject an effective amount of a hematopoietic stem or progenitor cell (HSPC) or a population of HSPCs to the subject. The HSPC or population thereof are in one aspect “donor” or donor-derived HSPC or a population thereof in that they are delivered or donated to the subject.

Applicant provides methods for reducing promoting or differentiating microganglia by administering to the subject an effective amount of a hematopoietic stem or progenitor cell (HSPC) or a population of HSPCs to the subject. The HSPC or population thereof are in one aspect “donor” or donor-derived HSPC or a population thereof in that they are delivered or donated to the subject.

Applicant provides methods for promoting the differentiation of a donor hematopoietic stem or progenitor cell or a donor cell population comprising the hematopoietic stem or progenitor cell into microglia, by administering to the subject an effective amount of a hematopoietic stem or progenitor cell (HSPC) or a population of HSPCs to the subject. The HSPC or population thereof are in one aspect “donor” or donor-derived HSPC or a population thereof in that they are delivered or donated to the subject.

Applicant provides methods for reducing neuroinflammation by administering to the subject an effective amount of a hematopoietic stem or progenitor cell (HSPC) or a population of HSPCs to the subject. The HSPC or population thereof are in one aspect “donor” or donor-derived HSPC or a population thereof in that they are delivered or donated to the subject.

In each of these methods, the HSPC or the population of HSPCs is wild-type. In another aspect the cells are CD34⁺ HSPCs. The cells in the population can be substantially homogenous HSPC or a clonal population, e.g., CD34⁺ HSPCs. They can be autologous or allogenic to the subject being treated. Methods to prepare substantially homogenous or clonal populations are known in the art.

In another aspect, the population of the HSPC cells comprises at least 80%, or at least 90%, or at least 95%, or at least 97%, of the cells of the population are CD34+ HSPCs.

The HSPC or population thereof can be administered as a composition, with one or more pharmaceutically acceptable carriers.

The methods can be combined with other therapeutic or diagnostic methods known in the art or as described herein. One of skill in the art can monitor the therapy and based on clinical or observational criteria, evaluate when the therapy has been effective. Effective therapy will differ with the subject being treated and the purpose of the therapy.

In a further aspect, the HSPC or a population of HSPCs is autologous or allogeneic to the subject.

In each of these methods, the subjects to be treated can be mammals, e.g., a murine, canine, feline, bovine, equine or a human patient. When the subject is a non-human mammal, the method is useful for the treatment of the non-human mammal or for use as an animal model to test new combination therapies. As is apparent to the skilled artisan, a control animal not receiving the treatment can be used as a point of reference.

Also provided is a kit comprising a genetically modified HSPC that comprises one or more of an exogenous or wild-type gene selected from amyloid beta precursor protein (APP), microtubule associated protein tau (MAPT), the gene encoding presenilin 1 (PSEN1) or triggering receptor expressed on myeloid cells 2 (Trem2), and instructions for use.

### Experimental

Without being bound by theory, Applicant hypothesized that wild-type (WT) HSPC transplantation could result in the generation of healthy microglia that could lead to the reduction of neuroinflammation and Aβ plaque build-ups in the most affected areas of the brain in AD. To explore this hypothesis, Applicant used the 5xFAD double transgenic mouse model, which expresses mutant human amyloid beta (A4) precursor protein 695 (App) with the Swedish (K670N, M671L), Florida (1716V), and London (V717I) Familial Alzheimer's Disease (FAD) mutations and human presenilin 1 (Psen1) harboring two FAD mutations (M146L and L286V). Aβ plaque accumulation is observed in the hippocampus and cortex of the 5xFAD mice by 2 to 4 months of age^{13,14}. These mice exhibit significant neurocognitive impairment and altered anxiety behavior between 3 to 6 months of age¹⁵. Moreover, 5xFAD mice demonstrate major features of amyloid plaque pathology of AD, including dysfunctional microglia^{13,16}.

Here, Applicant demonstrate that single WT HSPC transplantation into adult 5xFAD mice led to the preservation of memory and neurocognitive performance, and to the reduction of the Aβ plaque burden in the hippocampus and cortex. Donor HSPCs differentiated into microglia with active amyloid plaque clearance potential while also leading to the reduction of neuroinflammation. This work opens new therapeutic avenues using HSPC gene therapy for the treatment of AD, neurodegenerative disorders and symptoms associated with each thereof.

### Materials and Methods

Animals. All the mice were on a C57BL6 background. Applicant used the 5xFAD transgenic mouse model, (Tg6799, C57BL6-Tg (APPSwFlLon, PSEN1*M146L*L286V) 6799 Vas, Jackson Laboratory)¹³. Standard PCR reactions were carried out to identify the mice expressing all 5 mutations using the following primers: 5′-ACCCCCATGTCAGAGTTCCT-3′ (Common forward), 5′-CGGGCCTCTTCGCTATTAC-3′ (Mutant Reverse), and 5′-TATACAACCTTGGGGGATGG-3′ (WT Reverse). GFP transgenic mice, ubiquitously expressing enhanced GFP complementary DNA under the control of the chicken beta-actin promoter, were used as donors for the HSPC transplantation experiments (C57B1/6-Tg (CAG-EGFP) 1Osb/J; 003291, Jackson Laboratory). Mice were maintained in a temperature-and humidity-controlled animal facility, with a 12-hour light/dark cycle and free access to water and food. Both male and female mice were used in all experiments. All mice were bred at the University of California, San Diego (UCSD) vivarium, and all protocols were approved by the UCSD Institutional Animal Care and Use Committee.

HSPC isolation, transduction, transplantation, and engraftment. Bone marrow cells were flushed from the femurs of 6- to 8-week-old 5xFAD, GFP transgenic, mice. HSPCs were isolated by immunomagnetic separation using an anti-Scal antibody conjugated to magnetic beads (Miltenyi Biotec). Scal⁺ HSPCs were directly transplanted by tail vein injection of 2×10⁶ cells resuspended in 100 ml of phosphate-buffered saline (PBS) into lethally irradiated (7 gray; X-Rad 320, PXi) 5xFAD mice. For mice receiving WT GFP⁺ HSPCs, bone marrow cell engraftment of the transplanted cells was measured in peripheral blood 2 months after transplantation; blood samples freshly harvested from the tails were treated with red blood cell lysis buffer (eBioscience) and subsequently analyzed by flow cytometry (BD Accuri C6, BD Biosciences) to determine the proportion of GFP⁺ cells.

Behavioral tests. Mice were tested at 6 months of age using three main behavioral tests to evaluate the neurocognitive and locomotor activity. Prior to behavioral tests, the mice were exposed to the testing apparatus and testing room in order to acclimate to the environment. Applicant then performed and analyzed NORT as previously published^{55,56}. In brief, all mice were freely exposed to two replicates of the same object for 10 minutes during the familiarization phase before returning to their home cage. 24 hours later, one object was kept as a familiar object, while another object was replaced with a novel object. The entire process was recorded via an overhead video camera, and object investigation time was recorded manually by blinded researchers. The elevated plus maze test is one of the most widely used tests for measuring anxiety-like behavior in mice. Applicant performed this EPM used the protocol as published before⁵⁷. In brief, the elevated plus maze has the shape of a “+” with two alternate open and two alternate closed arms extending from a central platform. Each mouse was placed onto the center field and was allowed to explore the maze for 5 min. Time spent in the open and closed arms was recorded. Each animal was placed in the center of an open field arena (50 cm×50 cm) and allowed to move freely for 30 minutes with movement being recorded via overhead camera. The footage is then analyzed by an automated tracking system (ANY-Maze).

Immunohistochemistry analyses.—Brains were collected from euthanized mice and fixed using 10% formalin, embedded in paraffin wax, and sectioned at 5 □m thickness by standard methods. Sections were deparaffinized and stained with the mouse anti-6E10 (1:30,000; 803001, Biolegend), rabbit anti-GFP (1:600; ab183734, Abcam) and rabbit anti-Iba-1 (1:3000; SAG4318, Wako) antibodies. Slides were stained on a Ventana Discovery Ultra (Ventana Medical Systems). Antigen retrieval was performed using CC1 (Tris-EDTA based; pH 8.6) for 40 minutes at 95° C. The primary antibodies were incubated for 32 minutes at 37° C. For 6E10, an additional rabbit-anti mouse IgG antibody (1:6000; ab133469, Abcam) was utilized to minimize mouse-on-mouse non-specific staining. All rabbit antibodies were detected using an HRP-coupled goat anti-rabbit (OmniMap system; Ventana, catalog #760-4315) incubated on the sections for 12 min at 37° C. and visualized using diaminobenzidine as a chromogen followed by hematoxylin as a counterstain. Slides were rinsed, dehydrated through alcohol and xylene, and cover slipped. Images were acquired by Keyence BZ-X710 digital microscope system for high-resolution stitching images of tissue sections. ImagePro Premier software (Media Cybernetics) was used for the quantification of Aβ plaque and Iba1.

Immunofluorescence and image acquisition. Sections were deparaffinized and are transferred to pre-warmed antigen retrieval solution at 95° C. for 30 minutes and cooled at room temperature for 20 minutes and incubated in blocking solution (0.25% Triton X-100 and 3% BSA in tris-buffered saline). Sections were then incubated overnight at 4° C. with the following primary antibodies: rabbit anti-GFP (1:200; ab290, Abcam), chicken anti-Iba1 (1:200; 234006, Synaptic Systems), and mouse anti-6E10 (1:250; 805701, BioLegend). The appropriate Alexa Fluor-conjugated secondary antibodies (Invitrogen) were used for the visualization of antigens. Images were acquired using a Leica SP8 confocal microscope for high-resolution stitching images of tissue sections. Confocal image stacks and 3D view image were analyzed with the Imaris software (Bitplane, Oxford Instruments).

Aβ₁₋₄₂ ELISA. Brain tissue was homogenized in 0.15% triton in PBS containing protease inhibitor cocktail with a homogenizer and centrifuged at 200,000×g for 20 min at 4° C. The supernatant was removed and defined as the soluble fraction. Guanidine—HCl was added to give 0.5 m (final concentration) before application to enzyme-linked immunosorbent assay (ELISA). The pellet was solubilized by sonication in a 6 m guanidine—HCl buffer containing protease inhibitor cocktail. The solubilized pellet was centrifuged at 200,000×g for 20 min at 4° C. and used as the insoluble fraction. The amounts of Aβ₁₋₄₂ in each fraction were determined by a sandwich amyloid beta 42 ELISA kit (KHB3441, Invitrogen).

RNA-Seq. Sample preparation. Hippocampus and cortex were dissected from brains using previously publish protocol⁵⁸, and frozen at −80° C. in RNA later. Total RNA was isolated using the RNeasy Tissue kit (Qiagen) according to the manufacturer's instructions. RNA was assessed for quality using an Agilent Tapestation 4200, and 50 ng of RNA with an RNA Integrity Number (RIN) greater than 8.0 were used to generate RNA sequencing libraries using the Illumina® Stranded mRNA Prep (Illumina) following manufacturer's instructions. The resulting libraries were multiplexed and sequenced with 100 base pairs (bp) Paired End reads (PE100) to a depth of approximately 25 million reads per sample on an Illumina NovaSeq 6000. Samples were demultiplexed using bcl2fastq Conversion Software (Illumina).

RNA-seq data analysis. RNASeq data were processed using Kallisto (version 0.45.0), Mus musculus genome GRCm38 Ensembl version 94 annotation (Mus_musculus GRCm38.94 chr_patch_hapl_scaff.gtf). Gene-level TPM values and gene annotations were computed using tximport and the biomaRt R package. A custom python script was used to organize the data and log reduced using log2(TPM+1). A Composite Signature Analysis was performed and to compute the composite score of a set of genes, genes were first normalized and averaged. Gene expression values were normalized according to a modified Z-score approach centered around StepMiner⁵⁹ threshold (formula=(expr-SThr)/3*stddev). The samples were ordered based on the final combined score. Applicant performed differential expression analysis using DESeq2⁶⁰. The false discovery rate was controlled by setting an adjusted P value threshold of 0.1.

Statistics. Applicant did not exclude any animals from the experiments.

Experimenters were blinded to the genotype of the specific sample to every extent possible. In all experiments, cohorts were age-matched and sex-balanced. Non-objective computational measurements such as immunostaining quantification by scanning devices did not require blinding. All data are presented as the mean±s.e.m., and the statistics and graphs were performed using GraphPad software Prism v.9.0. No statistical methods were used to predetermine sample sizes, but sample sizes were similar to those reported in previous publications with 5xFAD model³⁵. One-way analysis of variance (ANOVA) followed by Tukey's multiple comparisons was used to compare multiple groups. Statistical significance was set at P<0.05. For RNA-seq data, gene signature was used to classify sample categories and the performance of the multi-class classification is measured by ROC-AUC (Receiver Operating Characteristics Area Under The Curve) values. A color-coded bar plot is combined with a violin plot to visualize the gene signature-based classification. All statistical tests were performed using R version 3.2.3 (2015-12-10). Standard t-tests were performed using python scipy.stats.ttest_ind package (version 0.19.0) with Welch's Two Sample t-test (unpaired, unequal variance (equal_var=False), and unequal sample size) parameters. Multiple hypothesis correction were performed by adjusting P values with statsmodels.stats.multitest.multipletests (fdr_bh: Benjamini/Hochberg principles). The results were independently validated with R statistical software (R version 3.6.1; 2019 Jul. 5). Violin, Swarm and Bubble plots are created using python seaborn package version 0.10.1.

Code Availability The codes used to conduct RNA sequencing analysis are publicly available at the following links: https://github.com/sahoo00/BoNE;

https://github.com/sahoo00/Hegemon.

### Results

Transplantation of WT Mouse HSPCs Results in Normal Neurocognitive Performance in 5xFAD Mice

To investigate the potential of WT HSPC transplantation for treating AD, Applicant intravenously injected lethally irradiated 2-month-old 5xFAD mice with Scal⁺ HSPC isolated from WT green fluorescent protein (GFP) transgenic mice (5xFAD/WT HSPC; n=10, 4 F and 6 M). Donor-derived HSPC engraftment was measured by flow cytometry, expressed as a percentage of GFP-positive blood cells in peripheral blood and ranged from 25 to 90%. As controls, Applicant analyzed WT littermates (WT; n=9, 5 F and 4 M), lethally irradiated WT littermate transplanted with WT mouse HSPC (WT/WT HSPC; n=10, 5 F and 5 M), non-treated 5xFAD littermates (5xFAD; n=10, 5 F and 5 M), lethally irradiated 5xFAD mice transplanted with 5xFAD HSPC (5xFAD/5xFAD HSPC; n=10, 5 F and 5 M).

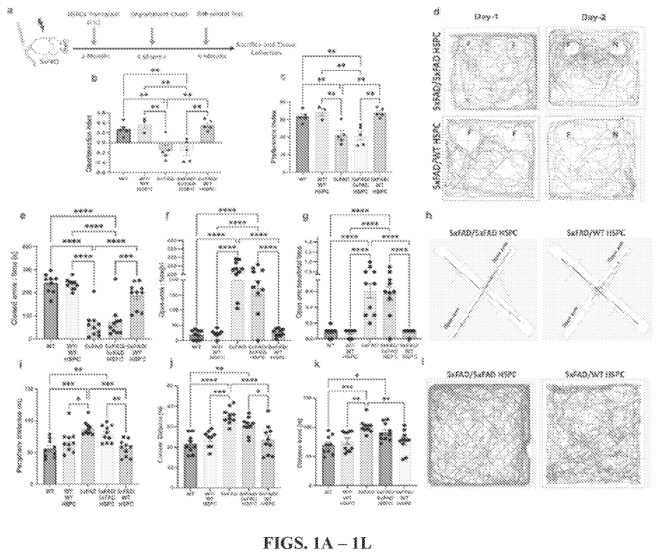

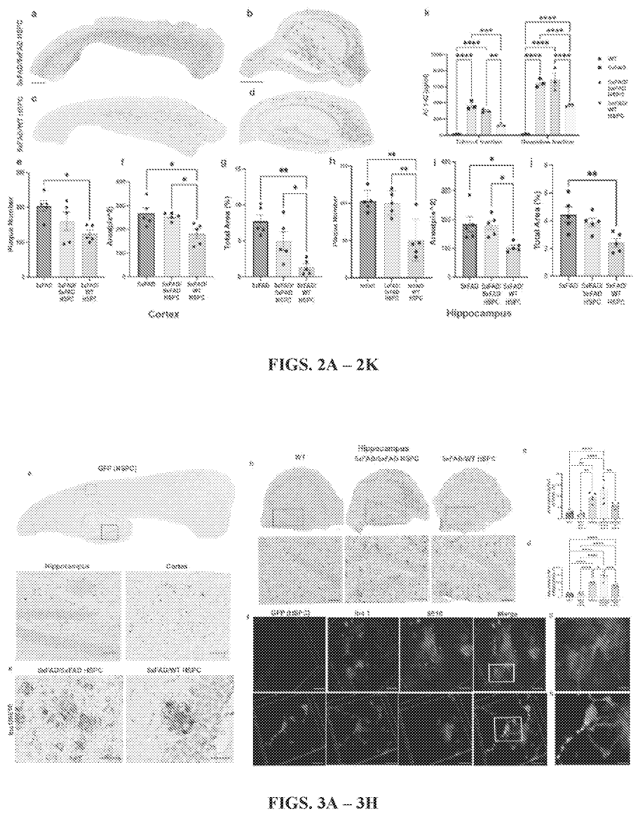

To determine the impact of WT HSPC transplantation on the neurocognitive function in the 5xFAD mice, Applicant performed a series of well-characterized behavioral testing in 6-month-old mice, i.e., 4 months post-transplantation. No difference between male and female was observed in any of the tests (data not shown). In addition, the myeloablative irradiation did not have any impact on activity in the WT and 5xFAD mice as no difference was observed in any of the tests when compared to WT/WT HSPC and 5xFAD/5xFAD HSPC, respectively (FIG. 1). Memory function of 5xFAD mice begins to deteriorate between 4-5 months of age^{17,18}. To determine whether WT HSPC transplantation prevents memory function loss, Applicant used a well-validated hippocampus dependent novel object recognition task (NORT)^{19,20}. Mice were briefly exposed to two identical objects (F1 and F2) during the training session, and in the test stage 24-h later, mice were exposed to a novel object (N) instead of F2; their discrimination indices were then calculated. On the day of habituation, the distance traveled by all group of mice were equivalent (data not shown). On the test day, WT, WT/WT HSPC, and 5xFAD/WT HSPC mice spent significantly more time exploring the novel object indicating that 5xFAD/WT HSPC transplanted mice remembered the familiar object and had a normal ability to discriminate between the novel and familiar objects. In contrast, the non-treated 5xFAD and 5xFAD/5xFAD HSPC transplanted mice showed no significant changes in the time exploring the novel or familiar objects (FIGS. 1B-1D), indicating impaired memory function.

Although impaired memory function are the hallmark features of AD, in addition it was shown that altered anxiety was also a significant complication and correlated with cognitive impairment in AD patients^{21,22}. Alteration in anxiety-like behavior was reported that 5xFAD mice in the elevated plus maze (EPM) correlated with spending more time in the open arms¹⁷. Indeed, the aversive nature of the open arms has been described as an innate fear of heights²³, and rodents prefer the closed arms because they provide protection from potential predation²⁴. Both 5xFAD and 5xFAD/5xFAD HSPC mice spent more time within the open exposed arm, suggesting an alteration in anxiety and perception of risks in these mice (FIGS. 1E-1H). In contrast, WT controls but also 5xFAD/WT HSPCs spent more time in the closed arms at a similar level (FIGS. 1E-1H). Next, Applicant tested the locomotor activity of the mice in an open field. General motor function as expressed as speed (data not shown) and distance were slightly elevated in 5xFAD and 5xFAD/5xFAD HSPC compared to WT, WT/WT HSPC, and 5xFAD/WT HSPC in the open field (FIG. 1K). In addition, 5xFAD and 5xFAD/5xFAD HSPC exhibited significantly higher level of peripheral/corner activity as compared to WT controls and 5xFAD/WT HSPC (FIGS. 1I-1L), showing preservation of normal anxiety-like behavior and activity in the 5xFAD mice treated with WT HSPCs. Altogether, these results demonstrate that transplantation of WT HSPC in 5xFAD mice lead to the preservation of their memory, perception of risks, normal anxiety level and locomotor activity.

Wild-Type HSPC Transplantation Decreased Amyloid-β Incumbrance in Brain of 5xFAD Mice

In AD, Aβ plaques are most abundant in the hippocampus and cortex areas of the brain¹⁵. Aβ deposition are also largely found in the hippocampus and cortex in 5xFAD mice at 6 months of age¹⁷. Applicant quantified the Aβ plaques in the hippocampus and cortex of 5xFAD mice after immunohistochemical analysis using the 6E10 antibody, which is directed against the amino acids 1-16 of Aβ. The level of Aβ in 5xFAD mice transplanted with 5xFAD HSPCs was equivalent to the non-treated 5xFAD mice in both hippocampus and cortex (FIGS. 2A-2B). In contrast, 5xFAD mice transplanted with WT HSPCs exhibited a significant reduction in plaque number, size and total area respectively in both cortex and hippocampus (FIGS. 2A-2J). These results prompted Applicant to test whether improved cognitive ability following reduction in plaque burden could be associated with a reduction in the level of soluble (Triton fraction) and insoluble (guanidine fraction) Aβ. Thus, in all group levels of Aβ₁₋₄₂ was measured by ELISA. Applicant found a reduction in the level of soluble and insoluble Aβ₁₋₄₂ following 4-month post transplantation with WT HSPC in 5xFAD mice compared to untreated and 5xFAD HSPC-treated mice (FIG. 2K). Thus, these results demonstrated that transplantation of WT HSPCs has a significant beneficial impact to reduce the Aβ plaque burden in cortex and hippocampus areas in 5xFAD mice.

Engrafted WT HSPCs Differentiate into Microglia and are Involved in Aβ Plaque Reduction in 5xFAD Mice

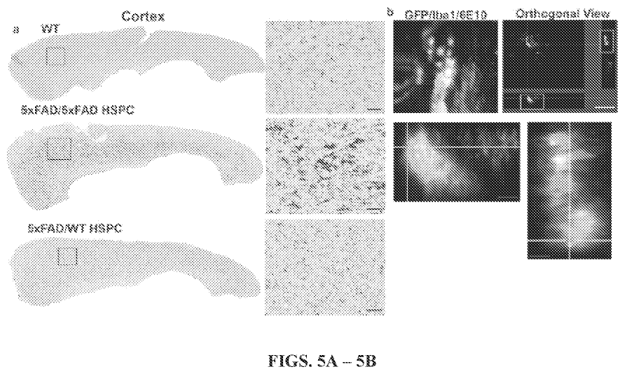

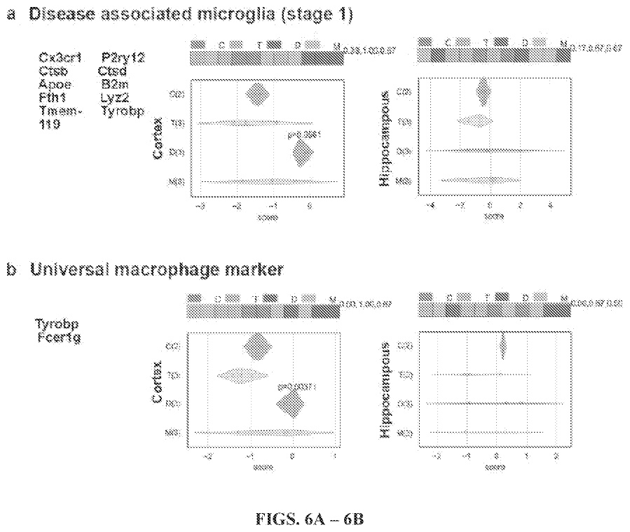

Applicant next investigated the engraftment and differentiation of WT GFP HSPCs in the brain of the 5xFAD mice and found substantial engraftment of GFP⁺ HSPC-derived cells within both the cortex and hippocampus of all treated mice (FIG. 3A). These cells were immunoreactive with Iba1, characterizing these cells as microglia-like cells (FIG. 3A, FIG. 3F). Recent reports indicated that microglia dysfunction contributes to the pathology of AD and is associated with Aβ plaque clearance^{25,26}. Both 5xFAD and 5xFAD/5xFAD HSPC mice exhibit significantly higher immunoreactive areas for Iba1 staining in the hippocampus and cortex compared to WT mice (FIG. 3C, FIG. 3D). In contrast, 5xFAD treated with WT HSPCs exhibited a significant reduction level of Iba1-positive cells in both hippocampus and cortex compared to the 5xFAD control groups. In addition, microglia were mostly ameboid and activated suggesting active inflammation in the hippocampus and cortex of the 5xFAD and 5xFAD/5xFAD mice (FIG. 3b) as previously described for AD^{25,26}. In contrast, the Iba1⁺ microglia in the 5xFAD/WT HSPC mice exhibited a more ramified “resting” phenotype (FIG. 3B), suggesting decreased inflammation in the brain of the treated mice. This was confirmed using dual chromogenic staining Iba1/6E10 to investigate the phenotype of the microglia in close proximity to the Aβ plaques. Plaque-associated Iba1⁺ cells were active and inflamed in 5xFAD mice transplanted with 5xFAD HSPC whereas they had a ramified and multipolar morphology in the brain of WT HSPC-transplanted 5xFAD mice, along with reduction in plaque size, mainly reflected by the clearance of the plaque halo (FIG. 3E). To prove that plaque associated Iba1⁺ microglia originated from the transplanted HSPC, Applicant performed immunofluorescence staining of brain sections from 5xFAD/WT HSPC mice and 5xFAD/5×FAD HSPC as control and observed Iba1⁺ microglia in contact with the plaques were also GFP⁺ (FIG. 3F). In addition, Aβ plaques were larger and more fibrous in 5xFAD controls compared to 5xFAD/WT HSPC mice, with a decrease in plaque size when surrounded by GFP⁺ microglia within 5xFAD/WT HSPC brain (FIG. 3F and FIG. 5). Altogether, these results suggested engulfment of amyloid plaques by microglia derived from the transplanted WT HSPC that cluster around the plaques and reduce the amyloid burden by actively phagocytosing amyloid fibers.

RNA Sequencing Analysis Reveals Major Differences in the Transcriptome Profile of Microglia in Cortex and Endothelial Cells in Hippocampus after WT HSPC Transplantation in 5xFAD Mice

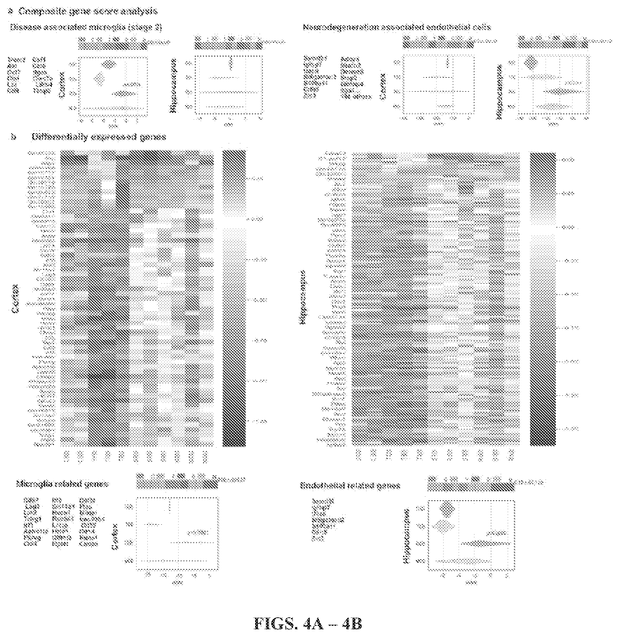

To decipher the relative contributions of HSPC transplantation to AD disease mitigation, Applicant examined the transcriptome profile by RNA sequencing (RNA-seq) of hippocampus and cortex of WT (n=2), 5xFAD (n=3), 5xFAD/5xFAD HSPC (n=3) and 5xFAD/WT HSPC (n=3) mice. Recent GWAS in AD patients have identified risk genes that are highly expressed in microglia and amplify the proinflammatory state^{6,27-29}. Since Applicant observed that transplanted WT HSPC differentiated into microglia in the brain leading to the decrease of inflammatory microglia in the hippocampus and cortex in 5xFAD mice, Applicant hypothesized diverse myeloid expression profile between the treated 5xFAD/WT HSPC mice and disease groups. Applicant therefore analyzed the transcriptome profile of the hippocampus and cortex by composite gene score analysis on previously published data set of signature genes associated with AD^{29,30}. Applicant evaluated two previously published sequential but distinct stages in “disease-associated microglia” (DAM) in AD. Stage 1 represents the first stage of DAM activation; it is Trem2-independent and involves activation of the set of genes Cx3cr1, P2ry 12, Tmem119, Tyrobp, Ctsb, Ctsd, Apoe, B2m, Fth1, Lyz2. The second phase of DAM activation, stage 2, includes induction of lipid metabolism and phagocytic pathway related genes which are associated with Aβ clearance (Trem2, AXL, Cst7, CtsI, LPL, Cd9, Csf1 Cc16, Itgax, Clec7a, Lirb4, Timp2)⁴. In addition to this, Applicant also assess previously published universal macrophage makers (Tyorbp, Fcer1g) for human and mice tissues, which were also found to be differentially expressed in AD³¹. Composite gene analysis revealed that stage 1 and stage 2 DAM markers and universal macrophage markers have higher expression levels in the cortex of 5xFAD and 5xFAD/5xFAD HSPC mice compared to 5xFAD/WT HSPC and WT mice because of an outlier in the 5xFAD/5xFAD HSPC, the difference was significant only with the 5xFAD mice (FIG. 4A, FIG. 6A, FIG. 6B). These data show that microglia inflammatory activation in 5xFAD mice is rescued by WT HSPC transplantation. This is confirmed by gene expression analysis (DEGs) that revealed that out of 61 genes differentially expressed in the cortex between 5xFAD/WT HSPC and 5xFAD mice, 49 were upregulated in 5xFAD compared to 5xFAD/WT HSPC mice, and 24 of them were related to microglia (FIG. 4B).

Interestingly, the microglia profile was not different between the different groups in the hippocampus. Further gene score analysis revealed a significant increase in expression of neurodegeneration associated endothelial cell genes in the hippocampus of 5xFAD mice and 5xFAD/5xFAD HSPC compared to 5xFAD transplanted with WT HSPC transplantation (FIG. 4A). DEG analyses also revealed that the top 7 differentially expressed genes in the hippocampus of 5xFAD/WT HSPC mice compared to 5xFAD controls are associated with endothelial cells (FIG. 4B). This phenomenon is specific to the hippocampus as the endothelial cell gene profile was not different in the cortex between the different group.

### Experimental Discussion

Alzheimer's disease is the most prevalent cause of dementia and the most common age-related neurodegenerative disorder. With an increasingly aging population, AD is projected to affect 131.5 million globally by 2050 and have a devastating impact. The costs of AD are accelerating from 1 trillion to a projected 2 trillion in 2030 which results in a heavy burden on patients, families, and the public-health system^{32,33}. Significant efforts have been made over the last several decades to create disease-ameliorating medicines that stop, prevent, or delay AD. Unfortunately, while hundreds of therapies from a variety of pharmacological classes have shown preclinical efficacy in animal models for alleviating cognitive impairment and disease load, none have yet proven effective in human clinical trials³³. Therefore, there is a pressing unmet medical need for the treatment of AD.

For a long time, reactive microglia have been considered as a consequence of AD pathology, however they are now regarded as potentially playing a role in disease progression and may be initiation^{25,27,29,34}. However, the roles of microglia in AD are still a matter of intense debate³⁵. Applicant's study represents direct evidence that microglia play a key role in disease progression and that replacing diseased App/Psen1 microglia with healthy ones via single WT HPSC transplantation in 5xFAD mice led to complete rescue of the neurocognitive impairment in the mice. Due to Aβ deposition being associated with cognitive changes, Applicant examined Aβ plaque levels in the different animal groups. In line with previous reports correlating Aβ reduction with functional and cognitive improvement, the effect of WT HSPC transplantation on behavior was accompanied by a reduction in the level of 6E10⁺ plaques and soluble Aβ₁₋₄₂ in the hippocampus and cortex. Without being bound by theory, Applicant hypothesized that WT HSPC-derived microglia directly assist with the clearance of toxic Aβ in the disease mouse model, in which microglia-mediated clearance pathways are dysregulated. In particular, earlier studies displayed that in amyloidosis mouse models and AD patients, plaques form over time^{36,37} and are surrounded by microglial cells, which seem to be reactive and inefficient in phagocytosing and clearing Aβ³⁸⁻⁴¹. In contrast, microglia derived from the transplanted WT HSPCs colocalized 6E10⁺ plaques that appear smaller, suggesting active engulfment of the Aβ plaques by the WT microglia in the 5xFAD/WT HSPC mice. This is consistent with a recent report that Trem2, which is involved in microglia-dependent phagocytosis, increases Aβ deposition is observed^{42,43}. In addition, it was shown that they could encase and form a barrier and phagocytose Aβ plaques when young and healthy²⁵, whereas microglia may participate in the spreading of Aβ aggregates to form new plaques by carrying and releasing phagocytosed Aβ seeds within the brain as observed by d'Erico et al³⁵. Applicant shows that this beneficial or detrimental action of microglia towards Aβ burden depends upon their ability to process the Aβ peptides and replacing App/Psen1-mutated microglia with WT HSPC transplantation can switch the balance towards decreasing the Aβ burden. Therefore, this data represents direct evidence of the involvement of the microglia into Aβ burden control and propagation.

Applicant noticed robust change in the phenotype and density of the microglia population after WT HSPC transplantation in 5xFAD. Proliferation and activation of microglia in the brain is an important characteristic in AD, and Applicant confirmed these features in the 5xFAD mice untreated or transplanted with App/Psen1 HSPCs. Remarkably, there was a distinct reduction in the number and change of morphology of Iba1⁺ microglia in WT HSPC-transplanted mice. This key feature also plays a direct role in neurocognitive preservation in 5xFAD mice. Indeed, chronically activated microglia continuously release inflammatory mediators and amplify the chronic inflammatory environment of AD, providing minimal protection and even contributing to synapse and neuronal loss by engulfment of synapses and secreted inflammatory factors injure the neurons^{9,41,44}. Applicant showed for the first time that replacing microglia carrying mutations in App and Psen1 genes by healthy microglia, led to decreased number of microglia and reduced inflammatory state. Applicant also showed that this was sufficient to prevent the neurocognitive impairments in the 5xFAD mice, strongly suggesting that microglia-associated neuroinflammation in AD is the main feature of neurodegeneration in AD and modulating this phenotype represents an important therapeutic target. Altogether, this data shows that healthy microglia have a therapeutic impact on AD by enhancing the engulfment of the plaques, limiting the spreading of the aggregates, and preventing chronic neuroinflammation.

To further evaluate changes in the gene profile of brain cell populations, Applicant examined the effects of WT HSPC transplantation on the cortex and hippocampus of 5xFAD mice using RNAseq analysis. It has been shown that microglia associated with plaques activate specific signaling pathway and Keren-Shaul et al. identified DAMs, which increase with disease progression in 5xFAD mice but also in human post-mortem brains²⁹. Indeed, DAM in AD are associated with the expression of genes, many of which were found in human genome-wide association studies (GWASs) including Trem2, a receptor required for DAM activation^{29,45}. In particular, DAMs are overexpressed significantly at later stages in AD, where the increased phagocytic and inflammatory activities lead to more damage to the surrounding cell populations. However, Applicant showed that 5xFAD mice transplanted with WT HSPCs exhibited a significant decrease of the DAMs in the cortex, while the newly introduced microglia cells retained the ability to phagocytize and engulf the plaques. The universal macrophage markers that increase in neurological diseases, including AD³¹, also decreased in the 5xFAD/WT HSPC mice. Overall, these data proved for the first time that pathways triggered by mutations in App and Psen1 genes can be prevented in the absence of these mutations in microglia.

Surprisingly, the microglia profile in the hippocampus did not differ between the different groups. But interestingly, the neurodegeneration associated endothelial cell gene expression profiling that has been reported as increased in other neurodegenerative disorders³⁰ has been also found to increase in this tissue compartment in 5xFAD mice, and this was corrected in the 5xFAD/WT HSPC mice. In AD, age dependent deterioration of the endothelial blood brain barrier (BBB) occurs in the human hippocampus, and severe impairment of the BBB transport mechanism has been reported in an advanced phase of disease and lead to hippocampus-dependent cognitive impairment^{46,49}. Furthermore, there is growing evidence from studies suggesting that vascular endothelial dysfunction plays a central role in the development of AD⁵⁰. In AD, accumulation of Aβ plaques may impair normal endothelial function and cause endothelial-dependent vasoconstriction⁵⁰. On the other hand, it has also been noted that endothelial cells engage in Aβ clearance when microglial clearance mechanisms are overwhelmed due to higher plaque burdens. Unlike microglia, however, endothelial cells do not proliferate efficiently. Consequently, persistent endothelial cell exposure to debris such as amyloid plaque initiates cell death, dysfunction, decreased blood flow, and increases the inflammatory response in AD⁵¹. Therefore, Applicant hypothesize that WT HSPC-derived microglia protect the endothelium in the hippocampus by clearing the plaques and decreasing inflammation, which further lead to AD rescue. These finding are in support of previous findings which claimed microglia and endothelial cells are crucial for maintaining cognitive function in AD⁵²⁻⁵⁴.

In conclusion, Applicant's findings demonstrate that transplantation of WT HSPCs into the 5xFAD mouse model resulted in the engraftment and differentiation of these cells into microglia to the rescue the AD disease phenotype. This report provides an answer to the long-standing debate of whether restoring microglial cells can modulate the Aβ burden during the course of disease progression and that this was enough to prevent the neurocognitive impairments in the 5xFAD mice, proving for the first time that microglia alone were able to modulate the Aβ burden and rescue AD. Therefore, this work strongly supports that this strategy is a therapeutic approach for treating AD and represents evidence for using HSPC gene therapy to correct known familial mutations in AD, opening new promising therapeutic perspectives for this incurable disorder.

### Incorporation by Reference

All publications, patents, and patent applications mentioned in this specification and attached Appendices are herein incorporated by reference to the same extent as if each individual publication, patent, patent application or appendix, was specifically and individually indicated to be incorporated by reference. Throughout the specification, Arabic numerals reference technical publications, the full bibliographic citations are provided in the reference section, immediately preceding the claims.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1 Sun, X., Chen, W. D. & Wang, Y. D. beta-Amyloid: the key peptide in the pathogenesis of Alzheimer's disease. Front Pharmacol 6, 221, doi:10.3389/fphar.2015.00221 (2015).
2 Jia, J. et al. The cost of Alzheimer's disease in China and re-estimation of costs worldwide. Alzheimers Dement 14, 483-491, doi: 10.1016/j.jalz.2017.12.006 (2018).
3 Robinson, M., Lee, B. Y. & Hane, F. T. Recent Progress in Alzheimer's Disease Research, Part 2: Genetics and Epidemiology. J Alzheimers Dis 57, 317-330, doi:10.3233/JAD-161149 (2017).
4 Vermunt, L. et al. Duration of preclinical, prodromal, and dementia stages of Alzheimer'sdisease in relation to age, sex, and APOE genotype. Alzheimers Dement 15, 888-898, doi:10.1016/j.jalz.2019.04.001 (2019).
5 Long, J. M. & Holtzman, D. M. Alzheimer Disease: An Update on Pathobiology and Treatment Strategies. Cell 179, 312-339, doi:10.1016/j.cell.2019.09.001 (2019).
6 Vaz, M. & Silvestre, S. Alzheimer's disease: Recent treatment strategies. European Journal of Pharmacology 887, 173554, doi:https://doi.org/10.1016/.eiphar.2020.173554 (2020).
7 Franco-Bocanegra, D. K. et al. Microglial morphology in Alzheimer's disease and after Abeta immunotherapy. Sci Rep 11, 15955, doi:10.1038/s41598-021-95535-0 (2021).
8 Fakhoury, M. Microglia and Astrocytes in Alzheimer's Disease: Implications for Therapy. Curr Neuropharmacol 16, 508-518, doi:10.2174/1570159X15666170720095240 (2018).
9 Cai, Z., Hussain, M. D. & Yan, L. J. Microglia, neuroinflammation, and beta-amyloid protein in Alzheimer's disease. Int J Neurosci 124, 307-321, doi:10.3109/00207454.2013.833510 (2014).
10 Hemonnot, A. L., Hua, J., Ulmann, L. & Hirbec, H. Microglia in Alzheimer Disease: Well-Known Targets and New Opportunities. Front Aging Neurosci 11, 233, doi:10.3389/fnagi.2019.00233 (2019).
11 Hansen, D. V., Hanson, J. E. & Sheng, M. Microglia in Alzheimer's disease. J Cell Biol. 217, 459-472, doi:10.1083/jcb.201709069 (2018).
12 Rocca, C. J. et al. CRISPR-Cas9 Gene Editing of Hematopoietic Stem Cells from Patientswith Friedreich's Ataxia. Mol Ther Methods Clin Dev 17, 1026-1036, doi:10.1016/j.omtm.2020.04.018 (2020).
13 Rocca, C. J. et al. Transplantation of wild-type mouse hematopoietic stem and progenitorcells ameliorates deficits in a mouse model of Friedreich's ataxia. Sci Transl Med 9, doi:10.1126/scitranslmed.aaj2347 (2017).
14 Gaide Chevronnay, H. P. et al. Hematopoietic Stem Cells Transplantation Can NormalizeThyroid Function in a Cystinosis Mouse Model. Endocrinology 157, 1363-1371, doi:10.1210/en.2015-1762 (2016).
15 Rocca, C. J. et al. Treatment of Inherited Eye Defects by Systemic Hematopoietic Stem Cell Transplantation. Invest Ophthalmol Vis Sci 56, 7214-7223, doi:10.1167/iovs.15-17107 (2015).
16 Yeagy, B. A. et al. Kidney preservation by bone marrow cell transplantation in hereditarynephropathy. Kidney Int 79, 1198-1206, doi:10.1038/ki.2010.537 (2011).
17 Syres, K. et al. Successful treatment of the murine model of cystinosis using bone marrowcell transplantation. Blood 114, 2542-2552, doi:10.1182/blood-2009-03-213934 (2009).
18 Oakley, H. et al. Intraneuronal beta-amyloid aggregates, neurodegeneration, and neuron loss in transgenic mice with five familial Alzheimer's disease mutations: potential factors in amyloid plaque formation. J Neurosci 26, 10129-10140, doi:10.1523/JNEUROSCI.1202-06.2006 (2006).
19 Richard, B. C. et al. Gene Dosage Dependent Aggravation of the Neurological Phenotypein the 5XFAD Mouse Model of Alzheimer's Disease. J Alzheimers Dis 45, 1223-1236, doi:10.3233/JAD-143120 (2015).
20 Jawhar, S., Trawicka, A., Jenneckens, C., Bayer, T. A. & Wirths, O. Motor deficits, neuronloss, and reduced anxiety coinciding with axonal degeneration and intraneuronal Abeta aggregation in the 5XFAD mouse model of Alzheimer's disease. Neurobiol Aging 33, 196e129-140, doi:10.1016/j.neurobiolaging.2010.05.027 (2012).
21 Wendt, S. et al. Changes in phagocytosis and potassium channel activity in microglia of 5xFAD mice indicate alterations in purinergic signaling in a mouse model of Alzheimer's disease. Neurobiol Aging 58, 41-53, doi:10.1016/j.neurobiolaging.2017.05.027 (2017).
22 Jawhar, S., Trawicka, A., Jenneckens, C., Bayer, T. A. & Wirths, O. Motor deficits, neuronloss, and reduced anxiety coinciding with axonal degeneration and intraneuronal Aβ aggregation in the 5XFAD mouse model of Alzheimer's disease. Neurobiol Aging 33, 196.e129-140, doi:10.1016/j.neurobiolaging.2010.05.027 (2012).
23 Cotel, M. C., Jawhar, S., Christensen, D. Z., Bayer, T. A. & Wirths, O. Environmental enrichment fails to rescue working memory deficits, neuron loss, and neurogenesis in APP/PSIKI mice. Neurobiol Aging 33, 96-107, doi:10.1016/j.neurobiolaging.2010.02.012 (2012).
24 Reed, J. M. & Squire, L. R. Impaired recognition memory in patients with lesions limitedto the hippocampal formation. Behav Neurosci 111, 667-675, doi:10.1037//0735-7044.111.4.667 (1997).
25 Wang, H., Ferguson, G. D., Pineda, V. V., Cundiff, P. E. & Storm, D. R. Overexpression of type-1 adenylyl cyclase in mouse forebrain enhances recognition memory and LTP. NatNeurosci 7, 635-642, doi:10.1038/nn1248 (2004).
26 Burke, A. D., Goldfarb, D., Bollam, P. & Khokher, S. Diagnosing and Treating Depressionin Patients with Alzheimer's Disease. Neurol Ther 8, 325-350, doi:10.1007/s40120-019-00148-5 (2019).
27 Kaiser, N. C. et al. Differences in anxiety among patients with early- versus late-onset Alzheimer's disease. J Neuropsychiatry Clin Neurosci 26, 73-80, doi:10.1176/appi.neuropsych. 12100240 (2014).
28 Mendez, M. F. Erratum: The Relationship Between Anxiety and Alzheimer's Disease. J Alzheimers Dis Rep 5, 563, doi:10.3233/ADR-219003 (2021).
29 Pentkowski, N. S., Rogge-Obando, K. K., Donaldson, T. N., Bouquin, S. J. & Clark, B. J.Anxiety and Alzheimer's disease: Behavioral analysis and neural basis in rodent models of Alzheimer's-related neuropathology. Neurosci Biobehav Rev 127, 647-658, doi:10.1016/j.neubiorev.2021.05.005 (2021).
30 Lister, R. G. Ethologically-based animal models of anxiety disorders. Pharmacol Ther 46,321-340, doi:10.1016/0163-7258 (90) 90021-s (1990).
31 Dawson, G. R. & Tricklebank, M. D. Use of the elevated plus maze in the search for novelanxiolytic agents. Trends Pharmacol Sci 16, 33-36, doi:10.1016/s0165-6147(00)88973-7 (1995).
32 Oblak, A. L. et al. Comprehensive Evaluation of the 5XFAD Mouse Model for PreclinicalTesting Applications: A MODEL-AD Study. Front Aging Neurosci 13, 713726, doi:10.3389/fnagi.2021.713726 (2021).
33 Daria, A. et al. Young microglia restore amyloid plaque clearance of aged microglia. EMBO J 36, 583-603, doi:10.15252/embj.201694591 (2017).
34 Biber, K. Reestablishing microglia function: good news for Alzheimer's therapy? EMBO J 36, 565-567, doi:10.15252/embj.201796627 (2017).

1. A method for treating a neurodegenerative disease, disorder or symptom thereof in a subject in need thereof, wherein the neurodegenerative disorder or symptom is selected from the group of:
a) alleviating or treating Alzheimer's Disease (AD) related to microglia inflammation;
b) reducing Aβ plaque burden in the hippocampus and cortex;
c) promoting or differentiating microganglia;
d) promoting the differentiation into microglia; or
e) reducing neuroinflammation;
comprising administering an effective amount of a hematopoietic stem or progenitor cell (HSPC) or a population of HSPCs to the subject, thereby treating the neurodegenerative disease, disorder or symptom in the subject.

2. The method of claim 1, wherein the HSPC is a wild-type HSPC or population of HSPCs.

3. The method of claim 1, wherein the HSPC or population of HSPCs is genetically modified to correct genes tied to the neurodegenerative disorder.

4. The method of claim 1, wherein neurodegenerative disorder is Alzheimer's Disease that is due to mutations in known causative genes, optionally amyloid beta precursor protein (APP), microtubule associated protein tau (MAPT), the gene encoding presenilin 1 (PSEN1) or triggering receptor expressed on myeloid cells 2 (Trem2).

5. The method of claim 1, wherein a population of HPSCs are administered to the subject and at least 80% of the HPSCs in the population are CD34+ HPSCs.

6. The method of claim 1, wherein the HSPC or population of HSPCs are autologous or allogeneic to the subject.

7. The method of claim 1, wherein the neurodegenerative disorder is Alzheimer's Disease and the HSPC or population of HSPCs comprises one or more of an exogenous or wild-type gene selected from amyloid beta precursor protein (APP), microtubule associated protein tau (MAPT), the gene encoding presenilin 1(PSEN1) or triggering receptor expressed on myeloid cells 2 (Trem2).

8. The method of claim 1, wherein the neurodegenerative disorder is Alzheimer's Disease and the HSPC or population of HSPCs are modified to correct for known familial mutations in one or more of a familial mutation selected from: APP, MAPT, PSEN1 or TREM2.

9. The method of claim 8, wherein the known familial mutations are corrected by gene addition, optionally by use of a vector, such as an adeno-associated viral vector or a lentiviral vector.

10. The method of claim 8, wherein the known familial mutations are corrected by a method comprising CRISPR/Cas9 technology.

11. The method of claim 1, wherein the neurodegenerative disorder is selected from disorientation, loss of bodily function, neuroinflammation or cognitive impairment.

12. The method of claim 1, wherein the HSPC or population of HSPCs are detectably labeled.

13. The method of claim 7, wherein the one or more of APP, MAPT, PSEN1 or Trem2 is detectably labeled.

14. The method of claim 1, wherein the HSPC or population of HSPCs are administered systemically or locally.

15. The method of claim 1, wherein the HSPC or population of HSPCs are administered through the hippocampus.

16. The method of claim 1, wherein the subject is a mammal.

17. The method of claim 16, wherein the mammal is a murine, canine, feline, bovine, equine or a human patient.

18. A kit comprising a genetically modified HSPC that comprises one or more of an exogenous or wild-type gene selected from amyloid beta precursor protein (APP), microtubule associated protein tau (MAPT), the gene encoding presenilin 1 (PSEN1) or triggering receptor expressed on myeloid cells 2 (Trem2), and instructions for use.
